(19) United States
(12) Patent Application Publication
(10) Pub. No.: US 2024/0307461 A1
(43) Pub. Date: Sep. 19, 2024

(54) Probiotic composition comprising Bacillus clausii UBBC-07 for modifying Gut microbiome in IBD patients

(71) Applicant: Unique Biotech Limited, Hyderabad (IN)

(72) Inventors: Ratna Sudha Madempudi, Hyderabad (IN); Jayanthi Neelamraju, Hyderabad (IN)

(21) Appl. No.: 18/430,704

(22) Filed: Feb. 2, 2024

(30) Foreign Application Priority Data
Feb. 2, 2023 (IN) 202341006683

(51) Int. Cl. A61K 35/742; A61P 1/00

(52) U.S. Cl. CPC A61K 35/742; A61P 1/00

(57) ABSTRACT

The current invention discloses the method of treating, preventing and/or ameliorating Inflammatory Bowel Disease in patients by administering a probiotic composition comprising Bacillus clausii. The current invention discloses a method for, prevention and/or treatment, and amelioration of symptoms of Inflammatory Bowel Disease by use of Bacillus clausii, which modulates the microbiome in the gastro-intestinal tract and decreases inflammation in the patients.

### FIELD OF INVENTION

The current invention relates to the field of probiotic formulation for alleviating symptoms of inflammatory bowel disease. The current invention more specifically relates to a method of preventing, ameliorating and treating IBD symptoms by using a probiotic formulation comprising Bacillus clausii strain.

### BACKGROUND

Inflammatory bowel diseases (IBD) are chronic disorders of unknown aetiology characterized by persistent mucosal inflammation at different levels of the gastrointestinal tract. Inflammatory bowel disease is one of the more frequent forms of dysbiosis diseases. Ulcerative colitis (UC) and Crohn's disease (CD) are the two main types of chronic inflammatory bowel diseases. They are mainly characterized by inflammation. Both the diseases may occur in adolescents and adults and affect men and women equally. Despite the similarity between the symptoms of these two diseases, there are some differences between the symptoms of CD and UC.

IBD, especially UC, affects both children and adults. UC affects approximately 2.6 million people in Europe and 1.2 million people in North America. About 25% of these patients are diagnosed before 18 years of age. UC often begins in adolescence and about 25% of patients with IBD are younger than 20 years.

Ulcerative colitis causes continuous mucosal inflammation that is restricted to the colon whereas Crohn's disease causes discontinuous transmural inflammation anywhere throughout the gastrointestinal tract, although it most frequently affects the terminal ileum. Most common intestinal lesions consist of mucosal ulcerations, bowel wall swelling and stricturing of the intestinal lumen. These chronic inflammatory lesions may cause symptoms such as diarrhoea, faecal urgency, abdominal pain and fever, as well as complications of variable severity including bleeding, intestinal obstruction, sepsis and malnutrition. It causes pain, diarrhea, fever, and other symptoms. In addition to the serious effect on the lower part of the small intestine, CD can also occur in parts of the digestive tract including the large intestine, stomach, oesophagus, or even mouth. Moreover, malnutrition is very common in CD because the small intestine is responsible for the absorption of nutrients, and CD damages the small intestine. More than 50% of people with CD suffer from folate and vitamin D deficiency, while more than 50% of people with UC suffer from iron deficiency (Ref L, Seyedian et al).

Although the main cause of the IBD has not yet been fully understood, the reports till now highlight the role of genetic and environmental factors which influence the immune responses affecting the gut. Unlike other inflammatory diseases, IBDs cannot be treated easily. IBD leads to erosion of the intestinal wall due to the inflammatory pathways triggered in IBD.

Several theories have been put forward for factors contributing to TBD, such as disruption of the intestinal mucous system which increases the immunological response rate in the human microbiota, changes in the content of the gut flora or the disruption of the epithelium function that stimulates the pathologic response in the normal mucous system, and also factors such as the patient's susceptibility, mucosal immunity, diet and lifestyle, and microflora of the intestine. It is generally accepted that the etiopathology of TBD is multifactorial, involving genetic predisposition, mucosal barrier dysfunction, disturbances in the gastrointestinal microbiota, dysregulated immune responses, environmental, and lifestyle factors (Ref 1 & 2, Seyedian & Cal et al respectively).

Human gut is an affluent and favorable niche for anaerobic bacteria. The major portion of gut microbiota are composed of strict anaerobes, which outnumber the facultative anaerobes and aerobes. These anaerobic gut bacteria play vital roles in human physiology and are essential for the maintenance of normal gut functions including nutrient absorption, metabolism and immune responses. Recognition of the important role of microbiome communities in human health and disease is a major paradigm shift in medical science. Gut microbiota composition is dynamic and individualized depending on the various environmental factors including geography, diet and life style. Alteration in the composition and proportions of microbiota is known as dysbiosis and that may lead to development of different diseases. Lactobacilli, Bifidobacteria, Akkermansia, Faecalibacterium, Bacteroides, Roseburia, Blauta, Eubacterium, Anaerostipes etc are among the commensal anaerobic bacteria of the human gut. An adequate colonization of anaerobes and a regulated cross-talk between these commensal bacteria and host system are required to maintain homeostasis. These anaerobic commensals are abundant in the healthy intestine but reduced in IBD which led to a disturbed mucosal homeostasis (Ref 3, Bamola et al).

Microbiota and human health relationship studies have supported the modulation of microbiota profile through administration of probiotics for the prevention and treatment of increasing number of diseases. Probiotics may restore the composition of the gut microbiome and introduce beneficial functions to host including prevention of gut inflammation. Probiotic bacteria exert their beneficial effects to the host by modulating metabolic activities, immunity and gut microbiota. Use of probiotics to maintain the microbial homeostasis is an important preventive and therapeutic approach for different diseases including inflammatory bowel disease (IBD).

The current invention relates to a method of preventing, treating and/or ameliorating the symptoms of IBD by administering a probiotic composition comprising Bacillus clausii. In some embodiments, the Bacillus clausii strain is the B. clausii UBBC-07 strain. The probiotic treatment can be given with standard medical treatment for IBD.

The current invention discloses a method of modulating the gut microbiota profile, and change cytokine secretion profile to reduce inflammation in IBD patients by treatment with this probiotic formulation comprising Bacillus clausii UBBC-07.

### SUMMARY

One embodiment of the current invention is a method for preventing, treating and/or ameliorating the symptoms of Inflammatory Bowel Disease (IBD) in a patient by modulating microbiome in Gastro-intestinal (GI) tract of the patient and decreasing inflammation in the patient, the method comprising the step of administering to the patient an effective dosage regimen of a probiotic composition comprising Bacillus clausii.

In one embodiment, the method disclosed herein, wherein the probiotic composition comprises a specific Bacillus clausii UBBC-07 strain of Bacillus clausii.

In one embodiment, each dose of the probiotic composition comprises 1-10 billion cfu of Bacillus clausii. In one embodiment, an effective dosage regimen of the probiotic composition is 1-2 doses a day administered every day for 4-12 weeks.

In one embodiment, each dose comprises 1-3 billion cfu of B. clausii. In one embodiment, each dose comprises 2 billion cfu of B. clausii. cfu

In one embodiment, an effective dosage regimen of the probiotic composition is 1-2 doses a day administered every day for 4-8 weeks.

In one embodiment, an effective dosage regimen of the probiotic composition is 2 doses a day administered every day for 4 weeks.

In one embodiment, the probiotic composition is administered to the patient as an adjunct to standard medical treatment (SMT) for IBD.

In one embodiment, the probiotic-composition is administered orally to the patient.

In one embodiment, administration of the effective dosage regimen of the probiotic composition to the patient ameliorates symptoms of ulcerative colitis and/or Crohn's disease in the patient.

In one embodiment, the amelioration of UC symptoms by administration of the probiotic composition disclosed herein comprises decrease in simple clinical colitis activity index (SCCAT) score in the patient.

In one embodiment, Bacillus clausii shows good survival in IBD patients in the patients administered the probiotic composition.

In one embodiment, the Bacillus clausii shows survival in the GI tract of at least 70% of the patients after administration of the probiotic composition.

In one embodiment, administration of the effective dosage regimen of the probiotic composition to the patient decreases Operational Taxonomic Units (OTiUs) of the phyla Bacteroides and increases OTUs of the phyla Firmicutes in the GI tract of the patient.

In one embodiment, the administration of the effective dosage regimen of the probiotic composition to the patient leads to an increase in OTUs of bacteria from at least one of the orders Lactobacillales, Erysipelotrichales and Bifidobacteriales; and leads to a decrease in OTUs of bacteria from at least one of the orders Bacteroidales or Selenomonadales in the GI tract of the patient.

In one embodiment, administration of the effective dosage regimen of the probiotic composition to the patient leads to an increase in OTUs of bacteria from at least one of the genera Lactobacilius, Bifidobacterium, Faecalibactcrium, Lachnospira, Blautia, and Alistipes and leads to a decrease in OTUs of bacteria from at least one of the genera Baceroides, Dialisier, Megasphaera, Roseburia, Olsenella or Meganomas in the GI tract of the patient.

In one embodiment, administration of the effective dosage regimen of the probiotic composition to the patient leads to an increase in OTUs of bacteria from at least one of the bacterial families Lactobacillaceae, Prevotellaceae, or Ruminococcaceae, and leads to a decrease in OTUs of bacteria from the families Lachnospiraceae, or Veillonellaceae in the GI tract of the patient.

In one embodiment, administration of an effective dosage regimen of the probiotic composition comprising the Bacillus clausii to the patient upregulates the serum levels of anti-inflammatory cytokines and decreases serum levels of pro-inflammatory cytokines in the patient.

In one embodiment, administration of an effective dosage regimen of the probiotic composition comprising Bacillus clausii to the patient upregulates the serum levels of anti-inflammatory cytokine IL-10 in the patient, and decreases serum levels of pro-inflammatory cytokines IL-6, IL-23, I1-17 and IL-11f levels in the patient.

In one embodiment, administration of the effective dosage regimen of the probiotic composition to the patient leads to increase in beneficial commensal bacteria in the GI tract of the patient.

In one embodiment, Bacillus clausii exhibits survival in the GI tract in at least 70% of the patients after oral administration of the probiotic composition.

### BRIEF DESCRIPTION OF FIGURES AND SEQUENCE LISTING

FIG. 1 shows analysis/abundance of the high abundant bacterial phyla in both the study groups, pretreatment and post treatment with Bacillus clausii UBBC-07.

FIG. 2. Shows analysis/abundance of the high abundance bacterial genera in both the study groups, pretreatment and post treatment with Bacillus clausii UBBC-07.

FIG. 3 shows analysis abundance of the low abundance bacterial genera in both the study groups, pretreatment and post treatment with Bacillus clausii UBBC-07.

SEQ ID NO: 1 is the forward primer for detecting Bacillus c/auisii UBBC-07 by amplifying 16S rRNA.

SEQ ID NO:2 is the reverse primer for detecting Bacillus clausii UBBC-07 by amplifying 16S rRNA.

SEQ ID NO:3 is the V3-V4 forward primer for metagenomic analysis of microbiome of IBD patients pre and post treatment with the probiotic composition comprising B. clausii UBBC-07.

SEQ ID NO:3 is the V3-V4 reverse primer for metagenomic analysis of microbiome of IBD patients pre and post treatment with the probiotic composition comprising B. clausii UBBC-07.

### DETAILED DESCRIPTION

The current invention relates to the use of a probiotic formulation for alleviating symptoms of inflammatory bowel disease. The current invention more specifically relates to method of preventing, ameliorating and/or treating IBD symptoms by using a probiotic formulation comprising Bacillus clausii.

The current invention relates to the use of a probiotic formulation for alleviating symptoms of inflammatory bowel disease. The current invention more specifically relates to method of preventing, ameliorating and/or treating IBD symptoms by using a probiotic formulation comprising Bacillus clausii, wherein the strain of B. clausii is B. clausii UBBC-07 strain.

Probiotics are defined as live microorganisms which, when administered in adequate amounts, confer a health benefit on the host. Bacterial spore formers, mostly of the genus Bacillus constitute are a main constituent of probiotic formulations being used currently. Bacilli being ubiquitous consistently enter the gastrointestinal and respiratory tracts of healthy people through food, ater, and air. Bacillus strains offer some advantages over the more common Lactobacillus products in that they can be stored indefinitely in a desiocated form without any deleterious effect on viability. In addition, they can also survive the low pH of the gastric barrier (Ref 4, Lakshmi et all Inflammatory bowel diseases (IBD) are chronic disorders of unknown aetiology characterized by persistent mucosal inflammation at different levels of the gastrointestinal tract. Inflammatory bowel disease is one of the more frequent forms of dysbiosis diseases. Alteration in the composition and proportions of microbiota is known as dysbiosis. Dysbiosis may lead to development of different diseases.

Ulcerative colitis (UC) and Crohin's disease (CD) are the two main types of chronic inflammatory bowel diseases. They are mainly characterized by inflammation. Both the diseases may occur in adolescents and adults and affect men and women equally. Despite the similarity between the symptoms of these two diseases, there are some differences between the symptoms of CD and UC.

Human gut is an affluent and favorable niche for anaerobic bacteria. The major portion of gut microbiota are composed of strict anaerobes, which outnumber the facultative anaerobes and aerobes. These anaerobic gut bacteria play vital roles in human physiology and are essential for the maintenance of normal gut functions including nutrient absorption, metabolism, and immune responses.

Human-associated bacteria largely belong to one of four phyla, Actinobacteria, Firmicutes, Proteobacteria, and Bacteroidetes. Mainly, the gut microbiota of healthy adults consists mainly of phyla Firmicutes, Bacteroidetes, and Actinobacteria. In addition, small amounts of Proteobacteria, Verrucomicrobia, Euryarchaeota, and Fusobacteria have also been found in human fecal samples.

Compared to healthy subjects, most studies of CD patients have reported decreased bacterial diversity accompanied by a reduction of protective gut microbiota, such as Firmnicutes (e.g., butyrate-producing bacteria Faecalibacterium, Roseburia, Oscillibacter and Coprococcus, etc.), Actinobacteria (e.g., Bifidobaclerium) and Ve-rrucomicrobia (e.g., the mucolytic bacteria Akkermansia mucinphila), combined with an expansion of putative inflammatory groups, such as Fscherichna coli (phylum Proteobacteria), Fusobacterium spp. (phylum Fusobacteria), and the species Ruminococcus gnavus (producer of an inflammatory polysaccharide).

Several studies have been conducted to understand the microorganisms affecting the development of IBD. Some studies have shown that the levels of Bacteroidetes in patients with CD is increased in comparison with healthy people, while Lactobacillus and Bifidobacterium are decreased. Also, the levels of enterobacteria increases significantly in patients with CD. On the other hand, patients with CD had higher antibody titers compared to E. Coli in healthy people (Ref 5, Bloom et al).

Recent findings have reported that gut dysbiosis (i.e., imbalance in the microbial community) may impact the integrity of the intestinal wall, increase the colonization of pathogenic bacteria, and also modulate the production of neurotransmitters, proinflammatory cytokines, neurotrophic factors, hormones, neuropeptides and tryptophan metabolism.

The main role of the intestinal barrier is in the digestion and absorption of nutrients; but it also controls the transport of antigens from the intestinal lumen into the submucosa, and maintains the balance between tolerance and the immune response to antigens that causes inflammation. The integrity of the intestinal barrier can be affected by diet, dysbiosis of the intestinal microbiota or by other factors. These have the potential to cause immune activation by translocation of microbial antigens and metabolites.

Although the main cause of the IBD has not yet been fully understood, the comprehensive studies carried out in this regard highlight the role of genetic and environmental factors. Many studies have also reported the role of genetic factors in the development of IBD. For example, various studies have highlighted the role of ethnic-racial differences in IBD, kinship, the prevalence of this disease in twins, chlorosomal relationships, hereditary and genetic factors, as well as genetic syndromes.

### Definitions

As used herein, the term “gut or Gastro-intestinal microbiota” refers to the vast number of microorganisms residing and proliferating in the human gastro-intestinal tract.

The human gut microbiota is a densely populated bacterial community with approximately 10¹¹ organisms per gram of fecal weight. It consists of over a 1000 species, most of which are obligate anaerobes, with a collective genome size 150-fold greater than that of its human host.

As used herein, the term “microbiota” and “microbiome” are used interchangeably, and refer to the compilation/collection of bacterial microorganisms within a specific environment. This term may also include their collective genomes.

As used herein, the terms “gut” and “gastro-intestinal tract” are used interchangeably herein.

As used herein, the term “inflammation” refers to the acute or chronic inflammation”.

As used herein the term “and/or” refers to occurrence of either one event, or of both events simultaneously.

As used herein, the terms “control/controlling” the “prevention/preventing”, “reduction/reducing”, “alleviation/alleviating”. “management/managing” or “mitigation/mitigating” means preventing, treating and/or improving/ameliorating the occurrence of symptoms that may be associated with IBD, stimulating regulatory cells of the immune system to inhibit inflammation.

As used herein, the “prevention” means preventing the occurrence of a disease. The “treatment” means suppressing the progress of a disease having occurred, alleviating a disease having occurred, and curing a disease having occurred.

As used herein, the term “patient” and “subject” are used interchangeably and refer to the person suffering and/or showing the symptoms of IBD, and also includes people who may not be exhibiting current symptoms of IBD but are predisposed to it.

As used herein the terms “patients” can include the following herein: Examples of the intended patients of the present invention can include
patients suffering from an inflammatory bowel disease (including related diseases for the present invention).
a patient with an inflammatory bowel disease for which the use of a steroid preparation, an immunosuppressant, or a biological product is difficult or insufficiently effective;
a patient with an inflammatory bowel disease, determined to be steroid-resistant; or
a patient with an inflammatory bowel disease, determined to be steroid-dependent.
A patient who is in remission stage after conventional treatment for IBD
A patient who is at increased risk for IBD due to lifestyle or genetic/family factors,
   As used herein, the terms “composition” and “formulation are used interchangeably.
   As used herein “effective dosage regimen” refers to the specific way a therapeutic drug is to be taken, including formulation, route of administration, dose, dosing interval, and treatment duration, so as to have the desired effect.

In the current invention “effective dosage regimen” of the multistrain probiotic formulation has the desired effect of preventing, treating and/or ameliorating OCD symptoms.

As used herein, the term “dose” refers to the measured and specific amount of a therapeutic composition, administered at one time. The number, and frequency of doses given over a specified period of time or prescribed intervals makes up the dosage regimen.

### Diagnosis of IBD:

The severe and the mild cases of IBD greatly differ in symptoms: the symptoms are intense in an active stage (advanced stage) during which inflammation is intense, and the symptoms abate in a remission stage during which the inflammation abates. However, the narrowing, perforation, and fistulae cannot be restored, and these symptoms do not often disappear in the remission stage. In addition, there are some related diseases, such as non-specific multiple small-bowel ulcer and bowel Behcet's disease, which not infrequently show intractable nature.

The disease state of ulcerative colitis is classified according to the diseased area and the course and severity thereof. It is classified into “proctitis type”, “left-side colitis type”, and “whole colitis type” by area and into “fulminant”, “severe”, “moderate”, and “mild” depending on clinical manifestation by severity. On the other hand, Crohn's disease can occur all over the alimentary tract; thus, its symptom is diverse and these symptoms are sometimes intermittently seen. It is classified by lesion area into “small bowel type”, whose lesions are present only in the small bowel, “large bowel type”, whose lesions are present only in the large bowel, and “small-bowel/large-bowel type”, whose lesions are present in both of these areas. It may be classified by lesion type into “inflammation type”, “narrowing type”, and “perforation type”; the latter shows more intractable nature and is clinically a more problematic type of disease state.

In both diseases clinical scoring systems like Harvey-Bradshaw-Index (HBI) or partial Mayo-Score (pMS) are employed in daily practice of specialized health centers. The subjective nature of these clinical scoring systems results to bias in the discrimination from non-inflammatory bowel conditions. In CD it includes symptoms that may be the result of small bowel bacterial overgrowth, bile salt diarrhea, or fibrotic strictures in the absence of inflammation.

The parameters that can be used for assessing some somatic symptoms of TBD are headache, heartburn, muscle stiffness, shakiness, sleep problem, procrastination, overwhelming, difficulty in completing work, nervousness, restlessness, poor concentration, feeling of depression, quick temper and trouble relaxing. These symptoms can be assessed based on scores. The rise in the score indicating an increase in the severity of the symptom and drop in the score indicating the decrease in the severity of symptoms.

Although there is no specific test for the diagnosis of CD or UC, some physical examinations, laboratory examinations, and endoscopy are needed for diagnosis. In some cases, inflammatory bowel disease can be diagnosed by accurate medical examinations such as stool testing, complete blood count (CBC), Barium X-ray imaging, radiological tests, sigmoidoscopy, colonoscopy, upper endoscopy, capsule endoscopy, and some other blood tests. CT enterography technique is also used as the initial imaging technique for appraising IBD, because of a combination of rapid scan time, high-resolution evaluation of intestinal and extra-intestinal disease manifestations and 24 hours availability in most hospitals. Accordingly, rectal examinations can be performed as well. Thus, in 20-80% of patients with CD, perianal skin tags, itchiness, or pain around the anus may be suggestive of inflammation, fistulization, or abscess around the anal area, which are quite common.

After observing initial symptoms of the disease, the endoscopic test can be performed to confirm the final diagnosis. The fecal calprotecin is also examined (Ref 6, Pathirana et al). Accordingly, an increase in the fecal calprotecin up to 81-91% can be suggestive of developing of the disease. On the other hand, an increase in the fecal calprotecin up to 89-98% can have serious implications. An increase in the lactoferrin up to 80% is alarming, suggesting the likelihood of IBD. In severe cases, rectal bleeding, sudden and severe colitis, and colon cancer may emerge (Ref 7, Dai et al). Another study showed that the risk of cancer is higher in people with UC who have a history of cancer in their family.

### Treatment of IBD:

Multi-dimensionality of the Inflammatory Bowel disease and uncertainty of the severity of the disease have complicated the diagnosis and treatment. Therefore, the objectives of the medical treatment include multiple facets such as clinical treatment and improvement of the individual's clinical condition, improving the quality of life, nutritional support for the patient, and restricting the patient's need for admission or surgery.

The goals of IBD therapy are to achieve clinical remission with subsequent endoscopic remission and/or resolution of inflammatory activity on cross-sectional imaging. For achieving these therapeutic targets various agents are currently available such as 5-aminosalicylic acid (5-ASA), corticosteroids, immunomodulators and biologics.

Hence the three main current types of TBD treatments are:
1) steroid therapy; 2) immunosuppressant therapy (including biological product therapy); or 3) operative therapy.

However, for the respective therapies, concerns exist about side effects due to the long-term administration of steroid, such as osteoporosis, hypertension, diabetes, glaucoma, and infection; side effects associated with immunosuppressants, especially susceptibility to infection (ease of infection) and digestive symptoms such as diarrhea; and risks and disadvantages such as large invasion (body burden) of operative therapy and frequent occurrence of diarrhea due to colostomy and after colostomy withdrawal.

The first step in treating IBD is pharmaceutical treatments. Corticosteroids, aminosalicylates, antibiotics, supportive medications and immunosuppressive dnigs are used to treat IBD. According to the American Therapeutic Association, aminosalicylates can be used to treat improved UC. For mild to moderate disease, aminosalicylates are proper selective drugs that can be used in various forms. Some antibiotics such as anoxicillin, ciprofloxacin, metronidazole, and azithromycin can improve the symptoms of CD. Drugs used to treat oral lesions in CD include sucralfate, carboxymethylene glucose, or hydrocortisone. Methotrexate can also be used in patients who cannot tolerate azathioprine and mercaptopurine. However, surgical treatment can be used in cases where drug treatments do not improve the symptoms of LBD.

Mesalamine as a nonsteroidal anti-inflammatory drug with drug class of 5-aminosalicyclic-acid derivative has been used for a long time and has a powerful effect in the remedy of IBD. This drug is really useful, well-tolerated and a safe drug for the remedy of ordinary ulcerative colitis that applied in most patients who suffer this disorder, Example of Standard medical treatment (SMT) for the current invention, that is given in combination with the probiotic formulation disclosed herein, includes, the following regimen of treatment, but can be any other standard regimen, which is accepted by physicians, and known to person of ordinary skill in the art:
5-amninosalicylic acid (5-ASA); Sulfasalazine (3 g/day) or Mesalamine 800 mg orally 3 times a day.

Aminosalicylates are a class of anti-inflammatory medications, that act on the colon and small intestine without suppressing the immune system. They are effective in inducing and maintaining remission in mild to moderate ulcerative colitis. They comprise a number of different preparations including mesalamine (Asacol®, Lialda®, Apriso®, Pentasa®), balsalazide (Colazal®), and sulfasalazine (Azulfidine®).

The approach to therapy of UC is based on severity of symptoms with frontline therapy being aminosalicylates. The intolerance of sulfasalazine is a restrictive factor, hence newer formulations have been developed which are free of the sulphur component and have better side-effect profile. These drugs are composed of 5-ASA, the active moiety of sulfasalazine, without the sulfapyridine carrier molecule that is usually the main cause of the side effects. Mesalamine is one of the 5-ASA based agents currently available and indicated for treatment of UC (Ref 8, 9, & 10, Ye & Van, William, and Bergman & Parkes, respectively).

### IBD and Inflammation:

Within the gastrointestinal mucosa, aberrations in both innate and adaptive immunity have been implicated in the immunopathogenicity of IBD. Cytokines, the soluble mediators of the inflammatory response, are secreted by a myriad of immune cell types and act to control the nature, effectiveness, severity, progression, and persistence of the immune reaction. The interleukins (ILs) are key constituents of the cytokine profile found in gastrointestinal mucosa in IBD and thus have been identified as potential future therapeutic targets.

Cytokines are a broad group of signalling proteins released into the bloodstream and causes vascular permeability, or the ability of molecules to pass through blood vessels. They modulate the functions of individual cells, and regulate processes taking place under normal, developmental and pathological conditions. The major cytokine receptor families include, type I cytokine receptors, type II cytokine receptors, TNF receptors, IL-1 receptors, tyrosine kinase receptors, and chemokine receptors.

Various proinflammatory cytokines are currently known to play an important role in the pathogenesis of IBD. Cytokines such as interleukin-6 (IL-6) and soluble IL-2 receptor (sIL-2R) have been shown to modulate the intestinal immune system by increasing the expression of adhesion factors on endothelial cells enabling transmigration of phagocytes and lymphocytes to sites of inflammation. IL-6 is a pleiotropic mediator and play s an important role in the pathogenesis of IBD. During inflammation and under the influence of IL-6, hepatocytes rapidly increase production of C-reactive protein (CRP). Notably, CRP is one of the best-studied inflammatory parameters in IBD patients. Cytokines are heterodimeric molecules, composed of 2 subunits, many of which are shared among cytokine family members, for example, IL-12 is composed of the p35 and p40 subunits, IL-35 is composed of the EB3 and p35 subunits, IL-27 is composed of the EB13 and p28 subunits, and IL-23 is composed of the p19 and p40 subunits.

IL-1β is a potent pro-inflammatory cytokine secreted from innate immune cells. It exerts a profound chemotactic role for neutrophil recruitment, stimulates effector innate cells such as dendritic cells and macrophages, and is also involved in the recruitment and activation of the adaptive T-cell response. IL-1β has been known to be expressed at higher levels in colonic biopsies from patients with IBD, produced in increased quantities from mononuclear cells in areas of actively inflamed colonic mucosa, hence it most probably acts in a proinflammatory manner exacerbating disease activity.

Some reports have provided evidence that animal models of colitis are ameliorated with IL-1 blocking strategies. Moreover, elevated IL1β levels that occurred in a transgenic mouse model were found to be associated with hyper susceptibility to colitis induction. Moreover, genetic variation in the IL1β gene associated with higher serum levels has been linked to a lowered response to biologic therapy in a small cohort of human patients and also to steroid dependence in UC, suggesting a potential influence on treatment outcome.

IL-6 is a pleiotropic cytokine that has a role in augmenting the innate immune response and moulding the adaptive immune response, via the enhancement of T-cell survival. Moreover, IL-6 level is increased in the colonic mucosa and serum of patients with IBD. It has also been show % n that IL-6 serum levels are correlated with disease activity in CD and serve as a predictive prognostic biomarker for disease relapse (Ref 11, McLean et al).

In early clinical trials as well as in animal models, IL-12 has been implicated as a major mediator of CD and IC diseases. The cytokine IL-23 shares the same p40 subunit with IL-12, and the anti-p40 mAbs used in human and mouse IBD studies neutralized the activities of both IL-12 and IL-23. IL-10-deficient mice spontaneously develop enterocolitis. It has been reported that IL-23 is essential for manifestation of chronic intestinal inflammation, whereas IL-12 is not. A critical target of IL-23 is a unique subset of tissue-homing memory T cells, which are specifically activated by IL-23 to produce the proinflammatory mediators IL-A7 and IL-6 (Ref 12, Yen et al).

IL-12 and IL-23 have both been implicated in the pathogenicity of IBD, bolstering the Th1 and Th17 cell response, respectively. Preclinical models have shown that blocking IL-12 in vivo ameliorated TNBS colitis.

Hence, drugs that target multiple cytokines are also attractive therapeutic candidates for IBD. For example, tofacitinib, which is a small molecule pan JAK inhibitor. JAKs are a family of cytoplasmic enzymes critical to downstream signaling of many cytokines after engagement with the common gamma chain transmembrane receptor.

Another example of multi-cytokine targeted therapy for IBD is Ustekinunab. Ustekinumab is a medication used to manage and treat a variety of inflammatory conditions, including inflammatory bowel disease. Ustekinumab is a human monoclonal antibody typically used to treat moderate to severe plaque psoriasis, psoriatic arthritis, moderate to severe Crohn disease, or moderate to severe ulcerative colitis (inflammatory bowel disease). Ustekinumab mediates the body's T-cell response by acting as an antagonist against interleukin-12 (IL12) and interleukin-23 (IL23) (Ref 13, Colquhoun & Kemp).

As used herein, pharmaceutically acceptable or appropriate carriers can be, but not limited to, organic or inorganic, solid or liquid excipient which is suitable for the selected mode of application such as oral application or injection, and administered in the form of a conventional pharmaceutical preparation. Such preparation includes solid such as tablets, granules, powders, capsules, and liquid such as solution, emulsion, suspension and the like. Said carrier includes starch, lactose, glucose, sucrose, dextrine, cellulose, paraffin, fatty acid glyceride, water, alcohol, gum arabic and the like. If necessary, auxiliary, stabilizer, emulsifier, lubricant, binder, pH adjustor controller, isotonic agent and other conventional additives may be added.

The term “conventional pharmaceuticals or compounds” as used herein in the context of “other conventional pharmaceuticals or compounds used to treat IBD and/or IBD symptoms refers to those pharmaceuticals or compounds that persons of skill in the art (including but not limited to physicians) conventionally use to treat the above mentioned condition/disorder/symptom”.

### EMBODIMENTS

The current invention relates to a method of preventing, treating and ameliorating the symptoms of IBD by administering a probiotic composition comprising Bacillus clausii to a patient.

The current invention discloses a method of modulating the gut microbiota profile in patients by treatment with this probiotic strain.

One embodiment of the current invention is a method for preventing, treating andl/or ameliorating the symptoms of Inflammatory Bowel Disease (IBD) in a patient by modulating microbiome of gastro-intestinal (GI) tract of a patient, and decreasing inflammation in the patient, the method comprising the step of administering to the patient an effective dosage regimen of a probiotic composition comprising Bacillus clausii.

In one embodiment, the method disclosed herein, wherein the probiotic composition comprises a specific Bacillus clausii UBBC-07 strain of Bacillus clausii.

In one embodiment, each dose of the probiotic composition comprises 1-3 billion cfu of Bacillus clausii.

In one embodiment, the effective dosage regimen of the probiotic composition is 1-2 doses a day administered every day for 4-12 weeks.

In one embodiment, an effective dosage regimen of the probiotic composition is 1-2 doses a day administered every day for 4-8 weeks.

In one embodiment, an effective dosage regimen of the probiotic composition is 2 doses a day administered every day for 4 weeks.

In one embodiment, the probiotic composition is administered to the patient as an adjunct to standard medical treatment (SMT) for IBD.

In one embodiment, the method of treatment disclosed herein leads to a significant decrease in the symptoms of IBD and improvement in the psychological parameters associated with IBD in patients, when given in combination with standard medical treatment for IBD; as compared to patients receiving only SMT.

In one embodiment, the probiotic composition is administered orally to the patient.

In one embodiment, Bacillus clausii shows good survival in IBD patients after oral administration,

In one embodiment, the Bacillus clausii shows survival in GI tract of at least 70% of the patients after administration of the probiotic composition.

In one embodiment, administration of the effective dosage regimen of the probiotic composition to the patient ameliorates symptoms of ulcerative colitis and/or Crohn's disease in the patient.

In one embodiment, the amelioration of UC symptoms by administration of the probiotic composition disclosed herein comprises decrease in simple clinical colitis activity index (SCCAI) score in the patient.

In one embodiment, administration of the effective dosage regimen of the probiotic composition to the patient decreases Operational Taxonomic Units (OTUs) of the phyla Bacteroides and increases OTUs of the phyla Firmicutes in the G1 tract of the patient.

In one embodiment, the administration of the effective dosage regimen of the probiotic composition to the patient leads to an increase in OTUs of bacteria from at least one of the orders Lactobacillales, Erysipelotrichales and Bifidobacteriales; and leads to a decrease in OTUs of bacteria from at least one of the orders Bacteroidales or Selenomnomadales in the GI tract of the patient.

In one embodiment, administration of the effective dosage regimen of the probiotic composition to the patient leads to an increase in OTUs of bacteria from at least one of the genera Lactobacillus, Bifidobacteriumm, Faecalibacterium, Lachnospira, Blautia, and Alistipes and leads to a decrease in OTUs of bacteria from at least one of the genera Bacteroides, Dialister, Megasphaera, Roseburia, Olsenella or Meganomas in the GI tract of the patient.

In one embodiment, administration of the effective dosage regimen of the probiotic composition to the patient leads to an increase in OTUs of bacteria from at least one of the bacterial families Lactobacillaceae, Prevotellaceae, or Ruminococcaceae, and leads to a decrease in OTUs of bacteria from the families Lachnospiraceae, or Veillonellaceae in the G1 tract of the patient.

In one embodiment, administration of an effective dosage regimen of the probiotic composition comprising the Bacillus clausii to the patient upregulates the serum levels of anti-inflammatory cytokines and decreases serum levels of pro-inflammatory cytokines in the patient.

In one embodiment, administration of an effective dosage regimen of the probiotic composition comprising Bacillus clausii to the patient upregulates the serum levels of anti-inflammatory cytokine IL-1 in the patient, and decreases serum levels of pro-inflammatory cytokines IL-6, IL-23, 11-17 and IL-1 levels in the patient.

In one embodiment, administration of the effective dosage regimen of the probiotic composition to the patient leads to increase in beneficial commensal bacteria in the GI tract of the patient,

In one embodiment, the Bacillus clausii exhibits survival in the G1 tract in at least 70% of the patients after oral administration of the probiotic composition to the patient.

One embodiment of the current invention is a method for treating, preventing or ameliorating at least one symptom of intestinal bowel disease, the method comprising the step of administering to a subject an effective dose of the probiotic formulation disclosed herein, in combination with SMT for IBD.

In one embodiment, the SMT for IBD given in combination with the probiotic formulation disclosed herein is 5-aminosalicylic acid (5-ASA)-Sulfasalazine (3 g/day) or Mesalamine 800 mg orally 3 times a day.

In one embodiment, each dose of the probiotic composition comprises 1-3 billion cfu of Bacillus clausii UBBC-07.

In one embodiment, an effective dosage regimen of the probiotic composition comprising Bacillus clausii UBBC-07 is 1-2 doses a day administered every day for 4-12 weeks.

In one embodiment, an effective dosage regimen of the probiotic composition is 1-2 doses a day administered every day for 4-8 weeks.

In one embodiment, an effective dosage regimen of the probiotic composition is 2 doses a day administered every day for 4 weeks.

In one embodiment, the probiotic composition comprising Bacillus clausii UBBC-07 is administered to the patient as an adjunct to standard medical treatment (S T) for IBD.

In one embodiment, the method of treatment disclosed herein leads to a significant decrease in the symptoms of IBD and improvement in the psychological parameters associated with IBD in patients, when given in combination with standard medical treatment for IBD; as compared to patients receiving only SMT.

In one embodiment, the probiotic composition comprising Bacillus clausii UBBC-07 is administered orally to the patient.

In one embodiment, administration of the effective dosage regimen of the probiotic composition comprising Bacillus clausii UBBC-07 ameliorates symptoms of ulcerative colitis and/or Crohn's disease in the patient.

In one embodiment, the amelioration of UC symptoms by administration of the probiotic composition comprising Bacillus clausii UBBC-07 disclosed herein comprises decrease in simple clinical colitis activity index (SCCAT) score in the patient.

In one embodiment, administration of the effective dosage regimen of the probiotic composition comprising Bacillus clausii UBBC-07 to the patient decreases Operational Taxonomic Units (OTUs) of the phyla Bacteroides and increases OTUs of the phyla Firmicutes in the GI tract of the patient.

In one embodiment, the administration of the effective dosage regimen of the probiotic composition comprising Bacillus clausii UBBC-07 to the patient leads to an increase in OTUs of bacteria from at least one of the orders Lactobacillales, Erysipelotrichales and Bifidobacteriales; and leads to a decrease in OTUs of bacteria from at least one of the orders Bacteroidales or Selenomonadales in the GI tract of the patient.

In one embodiment, administration of the effective dosage regimen of the probiotic composition comprising Bacillus clausii UBBC-07 to the patient leads to an increase in OTUs of bacteria from at least one of the genera Lactobacillus, Bifdobacterium, Faecalihbacterium, Lachnospira, Blautia, and Aistipes and leads to a decrease in OTUs of bacteria from at least one of the genera Bacteroides, Dialister. Megasphaera, Rosebiria, Olsenella or Meganomas in the (if tract of the patient.

In one embodiment, administration of the effective dosage regimen of the probiotic composition comprising Bacillus clausii UBBC-07 to the patient leads to an increase in OTUs of bacteria from at least one of the bacterial families Lactobacillaceae, Prevotellaceae, or Ruminococcaceae, and leads to a decrease in OTUs of bacteria from the families Lachnospiraceae, or Veillonellaceae in the GI tract of the patient.

In one embodiment, administration of an effective dosage regimen of the probiotic composition comprising Bacillus clausii UBBC-07 to the patient upregulates the serum levels of anti-inflammatory cytokines and decreases serum levels of pro-inflammatory cytokines in the patient.

In one embodiment, administration of an effective dosage regimen of the probiotic composition comprising Bacillus clausii UBBC-07 to the patient upregulates the serum levels of anti-inflammatory cytokine IL-10 in the patient, and decreases serum levels of pro-inflammatory cytokines IL-6, IL-23, 11-17 and IL-10 levels in the patient.

In one embodiment, administration of the effective dosage regimen of the probiotic composition comprising Bacillus clausii UBBC-07 to the patient leads to increase in beneficial commensal bacteria in the GI tract of the patient.

In one embodiment, the Bacillus clausii UBBC-07 exhibits survival in the GI tract in at least 70% of the patients after oral administration of the probiotic composition to the patient.

One embodiment of the current invention is a method for treating, preventing or ameliorating at least one symptom of intestinal bowel disease, the method comprising the step of administering to a subject an effective dose of the probiotic formulation disclosed herein, comprising Bacillus clausii UBBC-07, in combination with SMT for IBD.

In one embodiment, the SMT for IBD given in combination with the probiotic formulation disclosed herein is 5-aminosalicylic acid (5-ASA)-Sulfasalazine (3 g/day) or Mesalamine 800 mg orally 3 times a day.

The current invention encompasses a method of ameliorating symptoms associated with IBD, by treating subjects with a probiotic formulation comprising Bacillus clausii UBBC-07 in managing IBD.

In one embodiment, treatment with the probiotic formulation comprising the strain Bacillus clausii UBBC-07 positively affects the following states in IBD patients: (i) alteration in gut microbiota, (ii) alteration in concentration of serum cytokines, and (iii) alteration in serum concentration of dopamine and serotonin, (iv) improvement in disease symptoms, and (v) assessment of physical and psychological parameters.

In one embodiment, the probiotic treatment is given to the patients in a capsule form, as an oral dosage regimen.

The probiotic Bacillus sp. are in form of viable spores or in form of vegetative cells. The microbes are capable of germination and growth in the gut of the individual.

The probiotic formulation described in the invention is introduced into the gut of an individual by oral route in form of tablets, capsules powders or liquids. The microbes are resistant to the environment of the stomach and the small intestine, and hence can move through this environment and lodge themselves in the gut of the individual.

In one embodiment, the effective dose of the probiotic formulation alleviates IBD symptoms, when compared to placebo control. The placebo control may be receiving standard medical treatment for IBD concomitantly, without receiving the probiotic treatment.

In one embodiment, method of treating IBD symptoms by the probiotic formulation disclosed herein, encompasses the modulation of the gut microbiome and for changing cytokine profile to a lesser inflammatory profile. In one embodiment, this method encompasses treating gut dysbiosis in subjects or patients affected by TBD by the probiotic formulation disclosed herein, which helps increasing gut eubiosis.

In one embodiment, the probiotic formulation disclosed herein, comprises carriers, or excipients along with the B. clausii UBB-07 strain. In one embodiment, the probiotic formulation used for treating TBD further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb.

In one embodiment, the probiotic formulation disclosed herein, further comprises an additive, examples of which include, but are not limited to, saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

In one embodiment, the probiotic formulation is administered in combination with other therapeutic or ameliorative agents for treating the symptoms of IBD. The currently known pharmacological interventions for treating IBD are well known to a person of skill in the art, and are also described herein to some extent.

In one embodiment, probiotic formulation described herein and the at least one additional agent are administered as a single composition or formulation.

### Effect on Psychological Symptoms

In one embodiment, treatment with the probiotic formulation disclosed herein leads to amelioration of the different physical, behavioral and psychological symptoms. Symptoms can be assessed by scores, wherein a reduction in the score indicates the decrease in the severity of symptoms and rise in the score indicates the increase in the severity of the symptom.

In one embodiment, treatment with the probiotic formulation disclosed herein leads to amelioration of at least one, or more of the following class of symptoms associated with IBD, to different degrees: muscle stiffness, heartburn, headache, shakiness or tremor, difficulty in completing work, nervousness, depression, poor concentration, procrastination, restlessness, feeling of being overwhelmed, trouble relaxing, and Quick temper.

### EXAMPLES

The probiotic strain Bacillus clausii UBBC-07 (MTCC 5472) is a commercially available safe probiotic (Ref 3, Bamnola et al). The administration of this probiotic strain was reported safe and effective to improve the symptoms of acute diarrhea in children and adults (Ref 14, Li et al).

### Microbial Strain:

Bacillus clausii (B. clausii) UBBC-07 has been isolated by Unique Biotech Lid, India. It is well characterized and has been deposited in MTCC (Microbial Type Culture Collection), India under Indian Patent deposit with the number MTCC 5472. B. clausii UBBC-07 exhibits probiotic properties and being a spore forming probiotic, is stable over a wide range of temperatures with potential applications in a variety of formulations and foods (Ref 4. Lakshmi et al.,

A deposit of the Bacillus clausii UBBC-07 microbial strain has been made in a depository affording permanence of the deposit and ready accessibility thereto by the public if a patent is granted. The strains with their corresponding accession numbers is given below:
Bacillus clausii UBBC-07: MTCC 5472

The Microbial Type Culture Collection and Gene Bank (MTCC facility in IMTECH, Sector-39, Chandigarh, India-160036), is an affiliate member of the World Federation for Culture Collections (WFCC) and is registered with the World Data Centre for Microorganisms (WDCM).

The study for the effect of the probiotic strain Bacillus clausii UBBC-07 on TBD patients was registered with Clinical Trials Registry (CTRI)-India (CTRI/2019/11/022087) (Ref 15, Madempudi et al). Change in gut bacteria, serum cytokines, TBD symptoms, and disease severity were evaluated before and after intervention (Ref 3, Banrola et al).

### Example 1

Study design: A double blind, randomized, placebo-controlled study was conducted with intervention of 4 week to assess the efficacy of Bacillus clausii UBBC-07 (MTCC 5472) in IBD patient. The|randomization was generated by the nQuery clinical trial design sample size software in a 1:1 ratio. It was comprised of before intervention baseline visit (visit-1, week 0/day 0), and post intervention visit (visit-2, 1 week after completion of intervention).

Study site and ethical approval: The study was conducted in the tertiary care hospital after ethical approval from Institute Ethics Committee of All India Institute of Medical Sciences, New Delhi, India (e IEC.478/07.10.2016.OP-7) (Ref 3, Bamola et al). Before enrolment in the study, a written informed consent was obtained from each enrolled patient.

Subject selection: Adult patients of ulcerative colitis (UC) and Crohn's disease (CD) IBD of age between 18 and 60 years under standard medical treatment (SMT) were enrolled in the study. To quantify UC disease activity Simple Clinical Colitis Activity Index (SCCAI) score was used (Ref 16, Bewtra et al). SCCAI score is a validated symptom based clinical scoring index which is used by the clinicians to quantify UC disease activity and has a good correlation with disease activity indices. SCCAI score along with other symptoms of all the enrolled UC patients were assessed before and after intervention and UC patient with mild to moderate severity were included in this study.

The Crohn's Disease Activity Index (CDAT) is a validated scoring method to assess disease severity which has been developed and used for long (Ref 17, Shaoul et al). In CD severe disease is defined as a CDAI>450, moderate Crohn's is CDAI 220-450, mild Crohn's is CDAI 150-220, and clinic-al remission is defined as a CDAI<150, In this study the CD patient with mild to moderate severity were included 0.5-aminosalicylic acid (5-ASA)-Sulfasalazine (3 g/day) or Mesalamine 800 mg orally 3 times a day was SMT for the enrolled patient in this study.

Inclusion criteria: (a) Subject clinically diagnosed with Ulcerative Colitis (UC) or, Crohn's Disease (CD), (c) UC or CD patients of the age between 18 and 60-year of either sex (d) patient ready to give written consent to participate in the study, (e) subject receiving SMT and attending outpatient department (OPD) of the hospital.

Exclusion criteria: (a) Known case of any gastrointestinal disease other than IBD, (b) known case of any carcinoma (c) known case of immunodeficiency disorder, (d) patient is on any probiotic drug or consumed any probiotic in the last one month, (e) patient not taking food through oral route.

Sample size: SAS software was used to calculate the sample size. To detect the presence of a proportion difference, the assumption was made that a minimum of 116 subjects to be screened and 94 to be recruited to evaluate the primary endpoint. When the overall response is minimum 30% at a significant level of 0.05 this will provide 80% power to reject the null hypothesis.

Randomization: Subjects were randomized into two arms (Probiotic-Bacillus clausii UBBC-07-2 billion cfu twice a day or placebo twice a day). Randomization was conducted using opaque sealed envelopes that were indistinguishable between groups. The codes were kept blinded and given to the enrolled subjects based on the randomization numbers.

Enrolment of subjects: After screening as per inclusion and exclusion criteria, a total of 110 subjects were recruited, During baseline visit (day 0) complete medical history, medications, physical examination and vital signs including pulse rate, respiratory rate, blood pressure and temperature were assessed during hospital visit. FDA/DCGI/FSSAI approved probiotic strain Bacillus clausii UBBC-07 (MTCC 5472) was used as an intervention agent in this study. Enrolled subjects were given probiotic (Bacillus clausii UBBC-07, 2 billon-CFU/capsule) or placebo (identical to the probiotic capsule but contained only excipient, maltodextrin) twice in day for 4 weeks. The compliance of drug and dose were followed by observation and telephonic followup.

Data analysis: Data of 54 subjects were analyzed in each group. In probiotic treated group 19 patients of CD and 35 of UC were included. In placebo group 15 patients of CD and 39 of UC were included. The Stata statistical software (Version 14, USA) was used for statistical evaluations. Subject assessment was evaluated as frequency distribution and significance was assessed with chi-square test. A p value<0.05 was considered as statistically significant.

Outcome measures: The outcomes were measured by (i) detection of probiotic Bacillus clausii UBBC-07, (ii) alteration in gut microbiota, (iii) alteration in concentration of serum cytokines, (iv) alteration in serum concentration of dopamine and serotonin, (v) improvement in disease symptoms, (vi) assessment of physical and psychological parameters. For the evaluation of physical, behavioral and psychological parameters all the enrolled subjects were asked to answer the questionnaire as per the Hopkins Symptom Checklist (HSCL): A self-report symptom inventory (Ref 18, Derogatis et al). The parameters included were headache, heartburn, muscle stiffness, shakiness, sleep problem, procrastination, overwhelming, difficulty in completing work, nervousness, restlessness, poor concentration, feeling of depression, quick temper and trouble relaxing.

These symptoms were assessed based on scores. The rise in the score indicating an increase in the severity of the symptom and drop in the score indicating the decrease in the severity of symptoms.

Safety evaluation: Safety of the given probiotic was assessed by reporting of adverse event by the enrolled subject. During hospital visit, monitoring of vital signs including heart rate, respiratory rate, temperature and blood pressure were measured.

Sample collection and processing: A stool sample and a blood sample were collected from each enrolled subject before and after intervention. A fresh stool sample was collected in a sterile container and a blood sample in a plain vial was collected from each enrolled subject. After collection, the stool samples were aliquoted and processed for microbial identification and bacterial DNA isolation. Blood samples were also processed for serum separation. Serum was separated from the whole blood samples collected in the plain vial by centrifuging at 3000-4000 rpm for 5-10 mi. The serum samples were used for cytokine assays and stored at 80° C. till further use.

### Example 2: Survival of Bacillus clausii UBBC-07

Microbial detection: Mueller Hinton (MIH) broth, MH agar (Difco Laboratory, Detroit, MI) and Chrome Bacillus agar (Hi Media) were used to detect Bacillus strain. The stool sample were incubated for 24 h at 37° C. in MH broth and then plated on MH agar plate and Chrome Bacillus agar to isolate the Bacillus clausii UBBC-07. To detect Lactobacillus species de Man, Rogosa and Sharpe (MRS) broth and agar (Difco Laboratory. Detroit, MI) was used. The stool sample were incubated for 48 h at 37° C. in MRS broth in Anaerobic Glove Box (Anaerobic Workstation Whitley DG250-Don Whitley Scientific, United Kingdom) in anaerobic condition and then plated on MRS agar plate. Standard microbial culture and biochemical method and matrix assisted laser desorption ionization time of flight mass spectrometry (MALDI-TOF MS) (bioMdrieux Inc, USA) were used to identify the isolated colonies. The isolated organisms were also checked by molecular method.

Molecular detection: Bacillus clausii was checked by 16S rDNA sequencing using direct PCR using Thermal Cycler (Applied Biosystems, USA) with the in-house designed primers: forward 5′-CCTTGACGGTACCTCACCAC-3′(SEQ ID NO:1) and reverse 5′-AACCCCAATCTCTTGGGTGG-3′ (SEQ ID NO:2) with the product size 299 bp. The sequence similarity of primer was also checked using BLAST match (NCBI) which was 98%. After standardization bacterial DNA samples were amplified using standard PCR and PCR product was checked by the electrophoresis and Gel Doc System (BioRad, USA).

The presence of given probiotic was checked in the stool sample of the subjects of the both probiotic and placebo group after intervention. The stool sample wvas collected within one week of last dose of intervention given and checked for the presence of given probiotic strain. In both study groups the sample of the patients were evaluated by microbial culture method for the presence of B. clausii before and after treatment. No B. clausii was detected in both groups in the pre-treatment sample using microbial culture method. The presence of B. clausii was also checked and confirmed with conventional PCR using specific primers (SEQ ID NO:1 and SEQ ID NO:2). In the probiotic treated group, in the UC and CD patient B. clausii was detected in 74.5% and 79.6% of the subjects respectively and the detection of given probiotic in UC and CD patients of treatment group was significant (p<0.001) as compared to total absence or nil detection of B. clausii in placebo group. These results indicated the survival of orally given probiotic strain B. clausii UBBC-07 in GI tract of these subjects.

### Example 3: Metagenomic Analysis

DNA isolation for metagenomic analysis: Enrolled patients were asked to collect a fresh stool sample in provided sterile container on both pre and post intervention visit. Total bacterial DNA from stool sample was extracted using QIAamp DNA Stool Mini Kit (Qiagen) with some modification (Ref 19, Lim et al). Extracted DNA samples were checked and quantified by Nanodrop (TECAN Nano quant) and DNA samples oft C patients were processed for metagenomic analysis, Sequencing: Metagenomic analysis was performed on Illumina MiSeq® sequencing system (Illumina, San Diego, CA, USA). The analysis was carried out as per reported methodology (Ref 20, Tawik et al). V3-V4, hyper variable regions of 16S rRNA were amplified by V3-V4F (CCTACGGGNGGCWGCAG) (SEQ ID NO:3) and V3-V4R (GACTACHVGGGTATCT AATCC) (SEQ ID NO:4) primers. PCR amplification of DNA was carried out by KAPA HiFi HotStart Ready Mix as per standard protocol. The amplicons were purified using Ampure beads to remove unused primers and product was amplified with illumina primers to generate sequencing libraries. Qubit dsDNA assay kit and Illumina Miseq with 2×300PE sequencing kit were used for libraries preparation and sequencing (Ref 21, Nakayarna et al). Fast QC and Multi QC were used to check sequence data quality. Only QC passed reads were used in mothur for pairing and a known reference and UCHIME algorithm was used for chimeric sequence identification. Operational Taxonornic Unit (OTUs) and abundance was calculated using Silva v.132 database. Chaol and ACE, Shannon, Simpson, and Fisher indices were used for richness and relative abundance. To assess the difference among OTUs abundance between groups, Kruskal-Wallis rank surn test was used.

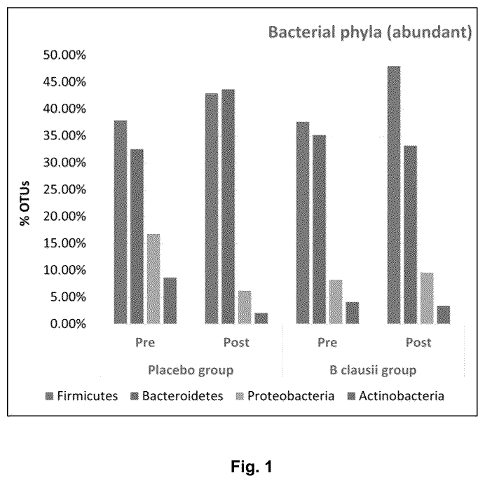

Before and after intervention, Operational Taxonomic Units (OTUs) were estimated for various bacterial taxa, including phylum, class, orders, families, and genera in the UC patients of both the probiotic and placebo groups. Firmicutes, Bacteroidetes, Proteobacteria, Actinobacteria, Cyanobacteria, and Euryarchaeota were abundant in both study groups. In the treatment group, the average % OTUs of the phylum Firmicutes were 37.58% and 48.0% in the before and after intervention samples, respectively and in the treatment group's post-intervention sample, the abundance of the phylum Firmicutes increased significantly. In the treatment group, the average % OTUs of the phylum Bacteroidetes were 35.13% and 33.14% in before and after intervention samples, respectively. In post-intervention samples, the treatment group showed a drop in OTUs of the phylum Bacteroidetes. Results are summarized in FIG. 1.

Among bacterial classes no significant change in OTUs was observed in before and after intervention samples in the treatment group. In the post-intervention sample. there was a drop in class Clostridia in the treatment group, and an increase in the placebo group. In the post-intervention samples of the treatment group, there was a drop in OTUs of the class Negativicutes and an increase in the placebo group. In the treatment group, OTUs of class Bacilli were 2.86% and 11.92% in pre and post intervention samples, respectively, while in the placebo group, the average percent OTUs of class Bacilli were 10.77% and 3.85% in pre and post intervention samples, respectively. There was a significant rise in OTUs of the class Bacilli in the treatment group's post-intervention samples and a drop in the placebo group.

Among bacterial orders in post-intervention samples in the treatment group, there was a drop in order Bacteroidales and an increase in the placebo group. In both groups, no significant changes in order Clostridiales were observed in post-intervention samples. After intervention there was a significant drop in order Selenomonadales in the treatment group and increase in the placebo group. In the treatment group, OTs of order Lactobacillales was 2.79% and 11.87% before and after intervention, respectively, and in the placebo group, 10.66% p and 3.82%<) before and after intervention, respectively. In post-intervention samples, there was a significant rise in the order Lactobacillales in the treatment group and a drop in the placebo group. In the treatment group, there was a significant rise in OTL's of the order Erysipelotrichales in postintervention samples. In post-intervention samples, there was an increase in order Bifidobacteriales in the treatment group and a drop in the placebo group.

Among bacterial families in both groups, there was an increase in OTUs of the Prevotellaceae in post-intervention samples. In postintervention samples in the treatment group, there was an increase in the family Ruminococcaceae, while in the placebo group, there was a decrease. In post-intervention samples, there was a decrease in the family Lachnospiraceae in the treatment group and an increase in the placebo group. There was a decrease in the family Veillonellaceae in the treatment group and an increase in the placebo group, in post-intervention samples. In the treatment group, OTUs of the family Lactobacillaceae were 2.12% and 6.03%, respectively, and 6.36% and 3.22% in before and after intervention, respectively. In post-intervention samples, there was a significant rise in the OTUs of Lactobaciliaceae in the treatment group and a drop in the placebo group. In post-intervention samples from the treatment group, there was an increase in the family Bifidobacteriaceae, while in the placebo group, there was a drop.

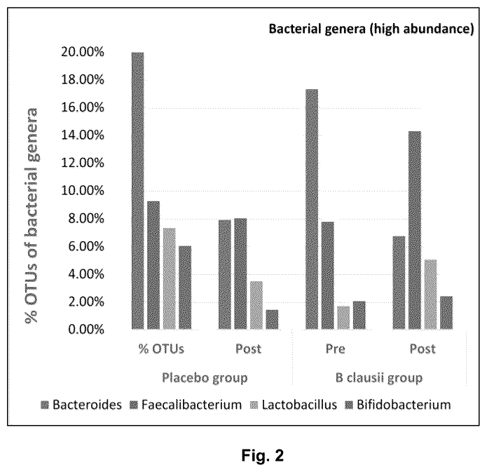

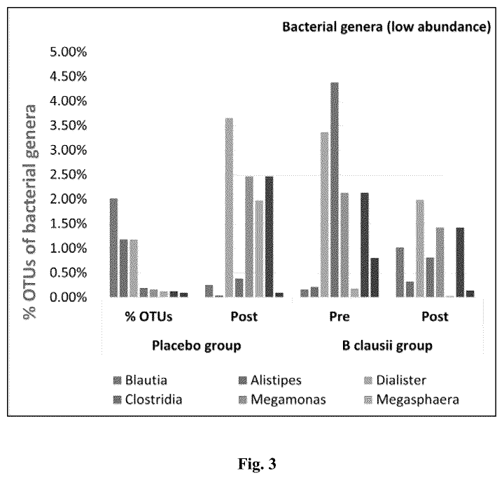

Among bacterial genera, the abundance of Lactobnciilus, Bifidobacterium, Faecalibacterium, Lachnospiraceae, Blautia, and Alistipes were increased and abundance of bacterial genera Bacteroides, Dialister, Megasphaera, Roseburia and Olsenella and Megamonas were decrease in the post intervention samples in the treatment group. In the post intervention samples an increase in genus Prevotella and decrease in genus Bacteroides were observed in both treatment and placebo group. Before and after intervention in the treatment group, the Faecalibacterium were 7.76% and 14.28% respectively, and in the placebo group, 9.23% and 8.01% respectively. There was a significant increase in OTUs of the genus Faecalibacterium in post intervention samples in the treatment group, while in the placebo group, there was a decrease in OTUs of the genus Faecalibacterium in post intervention samples. The average % OTUs of the Lactobacillus were 1.69% and 5.05% in the pre and post intervention samples in the treatment group, respectively, and 7.33% and 3.49% in the placebo group, respectively. In post-intervention samples, there was a significant rise in the genus Lactobacillus in the treatment group and a drop in the placebo group. Before and after intervention the OTUs of the Bifidobacterium were 2.05% and 2.41%, respectively in the treatment group and 6.03% and 1.42% before and post intervention, respectively in the treatment group. In postintervention samples, there was an increase in the genus Bifidobacterium in the treatment group and a drop in the placebo group. Results are summarized in FIGS. 1-3.

### Example 4: Cytokine Level Assessment

ELISA for Cytokines, serum serotonin and dopamine: A blood sample was drawn from each enrolled subject and serum was separated. The separated serum samples were assayed for the concentration of IL-10, TL-6, IL-1β, TNFα, 1L-17, 11-23, dopamine and serotonin as per manufacture's instruction (Fine Test, Fine Biotech Co. Ltd). In brief, (i) test samples and standards were added in 96 well coated plates, (ii) plates were incubated at 37° C. for 90 min and wells were washed with wash buffer, (iii) secondary antibody were added, (iv) plates were incubated at 37° C. for 60 min and wells were washed with wash buffer, (v) HRP was added (vi) plates were incubated at 37° C. for 30 min and multiple washings were done, (vii) TMB substrates were added to visualize HRP reaction, (viii) absorbance was recorded at 450 nm using Nanodrop, Nanoquant Infinite M 200 Pro-microplate reader (Texan, Austria GmbH).

Serum concentration of different cytokines were measured before and after intervention in the enrolled patients. Results indicated that the probiotic strain Bacillus clausii UBBC-07 was able to modulate the serum levels of cytokines in IBD patients. In the probiotic group serum IL-10 level were 21.6±4.8 pg/ml and 31.5±5.2 pg/ml pre and post intervention in UC patients (p<0.05) while the difference was not significant (p % ns) in the placebo group. In the CD patient in the probiotic treated group serum IL-10 level were 18.4±4.86 pg/ml and 28.4±4.9 pg/Ml pre and post intervention (p<0.05) while in the placebo group the difference was not significant (p % ns). In the treatment group serum IL-6 were 44.5±5.6 pg/ml and 31.5±4.4 pg/ml pre and post intervention in UC patient (p<0.05) while in the placebo group the difference was not significant (p W ns). In the UC patients, serum IL-17 level were 39±6.3 pg/ml and 24.5±4.5 pg/nl before and after intervention patient and the difference was significant (p<0.05) in the treatment group while in the placebo group the difference was not significant (p % ns). In the CD patient serum IL-17 level were 45.4±4.8 pg/nl and 28.6±7.4 pg/nil before and after intervention and the difference was significant (p<0.05) in the treatment group while the difference was not significant (p % ns) in the placebo group. Serum IL-23 were 898.5±54.6 ng/ml and 705.6±46.7 ng/nil pre and post intervention in UC patient in the treatment group (p<0.05) while the difference in the placebo group was not significant (p, ns). In the CD patient serum IL-23 level were 902.6±48.5 ng/ml and 734.6±40.8 ng/nl pre and post intervention in the treatment group (p<0.05) while in the placebo group the difference was not significant (p, ns). In the UC patient serum IL-1β) level were 384±45.5 pg/ml and 246±38.4 pg/nil before and after intervention in the treatment group (p<0.05) while the difference was not significant (p % ns) in placebo group. In the CD patient in the treatment group serum IL-1) level were 375.2±36.4 pg/nil and 268±32.2 pg/ml pre and post intervention (p<0.05) while in the placebo group the difference was not significant (p¼ ns). Results are summarized in Table 1.

### Example 5: Serum Serotonin and Dopamine Levels

Before and after intervention, serum serotonin levels were 125.8±17.6 ng/ml and 110.30±12.4 ng/nl, respectively, in the treatment group and 121.5: ±: 16.50 ng/nil and 106.8±14.65 ng/ml, respectively, in the placebo group no significant difference, Before and after intervention, serum Dopamine levels were 9.25±3.22 pg/nil and 10.45±3.5 pg/nl, respectively, in the treatment group and 10.50±2.6 pg/ml and 11.4±3.6 pg/nl, respectively, in the placebo group with no significant difference. There was no significant difference in serum serotonin and dopamine levels in treatment and placebo groups. Results are summarized in Table 1.

TABLE 1
Serum Cytokines, serotonin and dopamine levels in pre and post intervention
samples in treatment and placebo group.

| | Pre | Post | P value | Pre | Post | P value |
|---|---|---|---|---|---|---|
| UC patients | | | | | | |
| IL-10 | 21.6 ± 4.8 | 31.5 ± 5.2 | <0.05 | 23.5 ± 3.9 | 22.6 ± 7.5 | NS |
| IL-6 | 44.5 ± 5.6 | 31.5 ± 4.4 | <0.05 | 45.4 ± 6.2 | 40.6 ± 4.2 | NS |
| IL-17 | 39 ± 6.3 | 24.5 ± 4.5 | <0.05 | 38.8 ± 6.2 | 41.9 ± 4.6 | NS |
| IL-23 | 898.5 ± 54.6 | 805.6 ± 46.7 | NS | 906.8 ± 68.3 | 897.02 ± 73.5 | NS |
| IL-1b | 384 ± 45.5 | 246 ± 38.4 | <0.05 | 352.5 ± 67.6 | 386.7 ± 56.2 | NS |
| TNF-a | 65.6 ± 6.2 | 58.3 ± 5.6 | NS | 75 ± 5.2 | 68.5 ± 9.5 | NS |
| CD patients | | | | | | |
| IL-10 | 18.4 ± 4.86 | 28.4 ± 4.9 | <0.05 | 22.2 ± 4.1 | 25.6 ± 6.6 | NS |
| IL-6 | 44.4 ± 4.8 | 38.4 ± 7.2 | NS | 43.5 ± 5.6 | 46.7 ± 4.8 | NS |
| IL-17 | 45.4 ± 4.8 | 28.6 ± 7.4 | <0.05 | 41.4 ± 12.4 | 43.6 ± 6.8 | NS |
| IL-23 | 902.6 ± 48.5 | 834.6 ± 40.8 | NS | 911.5 ± 66.4 | 875.6 ± 56.5 | NS |
| IL-1b | 375.2 ± 36.4 | 268 ± 32.2 | <0.05 | 445 ± 58.2 | 284.5 ± 48.5 | <0.05 |
| TNF-a | 78.4 ± 6.2 | 65.3 ± 8.2 | NS | 66.2 ± 12.6 | 75.5 ± 10.8 | NS |
| Serum serotonin and dopamine in | | | | | | |
| both UC and CD patients | | | | | | |
| Serotonin (ng/ml) | 125.8 ± 17.6 | 110.30 ± 12.4 | NS | 121.5 ± 16.50 | 106.8 ± 14.65 | NS |
| Dopamine (pg/ml) | 9.25 ± 3.22 | 10.45 ± 3.5 | NS | 10.50 ± 2.6 | 11.4 ± 3.6 | NS |

### Example 6: Efficacy on Disease Symptoms

Data was collected and recorded for various symptoms of the disease as per standard protocol. Simple Clinical Colitis Activity Index (SCCAT) score was used to quantify UC disease activity (Ref 22, Bennebroek et al). The Crohn's Disease Activity Index (CDAI) score was used to assess CD (Ref 23. Freeman). Reduction in SCCAI score indicates the decrease in the severity of symptoms of UC and reduction in the CDAI score indicates the decrease in the severity of symptoms of CD, The SCCAI score in the UC patients was decreased in 42.52% in probiotic group and 31.58% in placebo group, after intervention with the difference being significant (p<0.05) between probiotic and placebo group. There was no significant difference in CDAI score of the CD patients between probiotic and placebo groups.

### Example 7: Physical, Behavioral and Psychological Parameters

Before and after intervention the enrolled IBD subjects were assessed for the different physical, behavioral and psychological symptoms. Symptoms were assessed by scores, reduction in the score indicates the decrease in the severity of symptoms and rise in the score indicates the increase in the severity of the symptom. There was no significant reduction in the complaint of muscle stiffness, heartburn, head ache, shakiness or tremor, difficulty in completing work, nervousness, depression, poor concentration after intervention in UC & CD patients in treatment group as compared to placebo group. The complaint of sleep problems was reduced in 4285% and 30.76% of UC patients in the treatment and placebo groups, respectively, with a significant difference between the groups (p 0.05). Sleep complaints were reduced in 36.84% and 26.66% of CD patients in the treatment and placebo groups, respectively, with a significant difference between the groups (p 0.05). Procrastination was reduced in 37.14% and 28.20% of UC patients in the treatment and placebo groups, respectively, with a significant difference between the groups (p<0.05). Procrastination was reduced in 36.84% and 26.66% of CD patients in the treatment and placebo group, respectively, with a significant difference between the groups (p<0.05). Restlessness was reduced in 28.20% and 20.51% of UC patients in the treatment and placebo groups, respectively, with a significant difference between the groups (p<0.05). Restlessness was reduced in 21.05% and 13.33% of CD patients, in the treatment and placebo groups, respectively, with a significant difference between the groups (p<0.05). Results showed significant (p<0.05) decreased in the complaint of sleep problem, complaint of procrastination, complaint of overwhelming, complaint of trouble relaxing, complaint of quick temper, compliant of restlessness in the post intervention in the probiotic treated group. Results are summarized in Table 2.

Safety evaluations: No adverse events were noticed, recorded, or reported during or after intervention in the trial, which further supported the safety of Bacillus clausii UBBC-07.

TABLE 2
Post intervention decrease in symptoms in the enrolled subjects for different
physical, behavioural and psychological parameters

| | Probiotic | Placebo | P | Probiotic | Placebo | P |
|---|---|---|---|---|---|---|
| | group | group | value | group | group | value |
| Procrastination | 37.14% | 28.20% | <0.05 | 36.84% | 26.31% | <0.05 |
| Restlessness | 28.20% | 20.51% | <0.05 | 21.05% | 13.33% | <0.05 |
| Muscles stiffness | 40.0% | 35.89% | NS | 36.84% | 40.0% | NS |
| Heartburn | 42.85% | 33.33% | <0.05 | 36.84% | 26.66% | <0.05 |
| Headache | 37.14% | 35.89% | NS | 31.57% | 26.66% | NS |
| Shakiness or tremor | 26.31% | 26.66% | NS | 42.85% | 30.76% | <0.05 |
| Sleep problem | 42.85% | 30.76% | <0.05 | 36.84% | 26.66% | <0.05 |
| Difficulty in completing work | 34.28% | 30.76% | NS | 31.57% | 33.33% | NS |
| Overwhelming | 37.14% | 25.64% | <0.05 | 31.57% | 20.0% | <0.05 |
| Trouble relaxing | 34.28% | 23.07% | <0.05 | 31.57% | 13.33% | <0.05 |
| Nervousness | 31.42% | 30.76% | NS | 36.84% | 13.33% | <0.05 |
| Depression | 25.71% | 25.64% | NS | 21.01% | 21.01% | NS |
| Poor concentration | 25.64% | 28.20% | NS | 31.57% | 26.31% | NS |
| Quick temper | 28.20% | 20.51% | <0.05 | 21.05% | 13.33% | <0.05 |

1. A method for preventing, treating and/or ameliorating the symptoms of Inflammatory Bowel Disease (IBD) in a patient by modulating microbiome in gastro-intestinal (GI) tract of the patient and decreasing inflammation in the patient, the method comprising the step of administering to the patient an effective dosage regimen of a probiotic composition comprising Bacillus clausii.

2. The method of claim 1, wherein the Bacillus clausii in the probiotic composition is the strain Bacillus clausii UBBC-07.

3. The method of claim 1, wherein each dose of the probiotic composition comprises 1-10 billion cfu of Bacillus clausii.

4. The method of claim 1, wherein each dose of the probiotic composition Comprises 1-3 billion cfu of Bacillus clausii.

5. The method of claim 1, wherein the effective dosage regimen of the probiotic composition is 1-2 doses a day administered every day for 4-12 weeks.

6. The method of claim 1, wherein the effective dosage regimen of the probiotic composition is 1-2 doses a day administered every day for 4-8 weeks.

7. The method of claim 1, wherein the probiotic composition is administered to the patient as an adjunct to standard medical treatment (SMT) for IBD.

8. The method of claim 1, wherein the probiotic composition is administered orally to the patient.

9. The method of claim 1, wherein administration of the effective dosage regimen of the probiotic composition to the patient ameliorates symptoms of ulcerative colitis and/or Crohn's disease in the patient.

10. The method of claim 9, wherein the amelioration of UC symptoms comprises decrease in simple clinical colitis activity index (SCCAI) score in the patient.

11. The method of claim 1, wherein administration of the effective dosage regimen of the probiotic composition to the patient decreases Operational Taxonomic Units (OTUs) of the phyla Bacteroides and increases OTUs of the phyla Firmicutes in the GI tract of the patient.

12. The method of claim 1, wherein administration of effective dosage regimen of the probiotic composition to the patient leads to an increase in OTUs of bacteria from at least one of the orders Lactobacillales, Erysipelotrichales and Bifidobacteriales; and leads to a decrease in OT Us of bacteria from at least one of the orders Bacteroidales or Selenomonadales in the GI tract of the patient.

13. The method of claim 1, wherein administration of the effective dosage regimen of the probiotic composition to the patient leads to an increase in OTUs of bacteria from at least one of the genera Lactobacillus, Bifidobacterium, Faecalibacterium, Lachnospiraceae, Blautia, and Alistipes and leads to a decrease in OTUs of bacteria from at least one of the genera Bacteroides, Dialister, Megasphaera, Rosehuria, Olsenella or Megamonas in the GI tract of the patient.

14. The method of claim 1, wherein administration of the effective dosage regimen of the probiotic composition to the patient leads to an increase in OT Us of bacteria from at least one of the bacterial families Lactobacillaceae, Prevotellaceae, or Ruminococcaceae, and leads to a decrease in OTUs of bacteria from the families Lachnospiraceae, or Veillonellaceae in the GI tract of the patient.

15. The method of claim 1, wherein administration of an effective dosage regimen of the probiotic composition comprising the Bacillus clausii to the patient upregulates the serum levels of anti-inflammatory cytokines and decreases serum levels of pro-inflammatory cytokines in the patient.

16. The method of claim 13, wherein administration of an effective dosage regimen of the probiotic composition comprising Bacillus clausii to the patient upregulates the serum levels of anti-inflammatory cytokine IL-10 in the patient, and decreases serum levels of pro-inflammatory cytokines IL-6, IL-23, 11-17 and IL-1β levels in the patient.

17. The method of claim 1, wherein administration of the effective dosage regimen of the probiotic composition to the patient leads to increase in beneficial commensal bacteria in the GI tract of the patient.

18. The method of claim 1, wherein the Bacillus clausii exhibits survival in the GI tract in at least 70% of the patients after oral administration of the probiotic composition.
